(19) United States
(12) Patent Application Publication
(10) Pub. No.: US 2007/0015214 A1
(43) Pub. Date: Jan. 18, 2007

(54) Methods for identifying compounds for regulating muscle mass or function using prostanoid IP receptors

(75) Inventors: Robert Joseph Isfort, Fairfield, OH (US); Russell James Sheldon, Fairfield, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(73) Assignee: The Procter & Gamble Company, Cincinnati, OH (US)

(21) Appl. No.: 11/487,848

(22) Filed: Jul. 17, 2006

Related U.S. Application Data
(60) Provisional application No. 60/700,292, filed on Jul. 18, 2005.

(51) Int. Cl. A61K 31/557; G01N 33/53

(52) U.S. Cl. 435/7.1; 514/573

(57) ABSTRACT

Methods for identifying compounds that bind to or activate prostanoid IP receptors and potentially regulate skeletal muscle mass or function are disclosed. Pharmaceutical compositions comprising IP receptor agonists and methods for increasing skeletal muscle mass or function or for the treatment of skeletal muscle atrophy using IP receptor as the target for intervention and methods for treatment of muscular dystrophies are described.

### CROSS REFERENCE TO RELATED APPLICATION

This application claims the benefit of U.S. Provisional Application No. 60/700,292, filed on 18 Jul. 2005, which is herein incorporated by reference in its entirety.

### FIELD OF THE INVENTION

The present invention relates to methods of identifying compounds for regulating skeletal muscle mass or function by regulating the activity of a prostanoid IP receptor. The invention also relates to methods for the treatment of skeletal muscle atrophy and/or methods for inducing skeletal muscle hypertrophy using a prostanoid IP receptor as a target for intervention and to methods of treating muscular dystrophies using the prostanoid IP receptor as a target.

### BACKGROUND OF THE INVENTION

Prostanoid IP Receptor and Its Ligands

Prostanoids that comprise of the prostaglandins (PG) and the thromboxanes (Tx), are cyclooxygenase products derived from C₂₀ unsaturated fatty acids. Prostaglandins exert a variety of actions in various tissues and cells, including, relaxation and contraction of smooth muscles, modulation of neurotransmitter release, regulation of secretions and motility in the gastrointestinal tract, regulation of the transport of ions and water in kidneys, immune system regulation, bone remodeling, and regulation of platelet aggregation, degranulation, and shape. They are also involved in apoptosis, cell differentiation, and oncogenesis (Narumiya et al. (1999) Physiol. Rev. 79, 1193-1226).

The prostanoids mediate their actions through specific receptors, many of which have been cloned and characterized. The prostanoid IP receptor is a high affinity receptor for PGI₂ and belongs to the family of G protein-coupled receptors (GPCRs). Agonist activation of the prostanoid IP receptor, amongst other effects, leads to activation of adenylate cyclase. Adenylate cyclase catalyzes the formation of cAMP, which in turn has multiple effects, including the activation of protein kinase A, intracellular calcium release, and activation of mitogen-activated protein kinase (MAP kinase).

Prostanoid IP receptors have been cloned from at least human, rat, and mouse. The prostanoid IP receptor mRNA expression has been reported from various tissues including neurons of the DRG, megakaryocytes, arteriole smooth muscle, kidney, spleen, and thymus. Similarly, the prostanoid IP receptor protein is expressed in various tissues including platelets, vascular smooth muscle, kidney, thymus, liver, lung, spleen, skeletal muscle, heart, neurons, and pancreas. Knockout mice lacking expression of prostanoid IP receptors have also been generated. These mice exhibit altered thrombotic tendency and decreased inflammatory swelling.

PGI₂ is the natural ligand/agonist of the prostanoid IP receptor. PGI₂ binds to and activates the prostanoid IP receptor. PGI₂ has several physiological functions including inhibition of platelet aggregation, vasodilation, and mediation of vascular permeability, suppression of gastric acid release, suppression of gastric/intestinal motility, anti-diuretic activities, and analgesia. Additional synthetic ligands have been described for the prostanoid IP receptor including but not limited to cicaprost, iloprost, beraprost, octimibate, carbaprostacyclin, carbacyclin, isocarbacyclin, ONO-1301, and BMY45778. The prostanoid IP receptor may be distinguished from other receptors pharmacologically using receptor selective agonists and antagonists.

Skeletal Muscle and Skeletal Muscle Atrophy and Hypertrophy

Skeletal muscle is a plastic tissue that readily adapts to changes in either physiological demand for work or metabolic need. Hypertrophy refers to an increase in skeletal muscle mass while skeletal muscle atrophy refers to a decrease in skeletal muscle mass. The loss or gain of muscle mass may lead to abnormal muscle function. Acute skeletal muscle atrophy is traceable to a variety of causes including, but not limited to: disuse, e.g., due to surgery, bed rest, or broken bones; denervation/nerve damage due to spinal cord injury; autoimmune disease, or infectious disease; glucocorticoid use; sepsis; nutrient limitation due to illness or starvation; or space travel. Skeletal muscle atrophy occurs through normal biological processes, however, in certain medical situations this results in a debilitating level of muscle atrophy. For example, acute skeletal muscle atrophy presents a significant limitation in the rehabilitation of patients from immobilizations. In such cases, the rehabilitation period required to reverse the skeletal muscle atrophy is often far longer than the period required to repair the original injury. Such acute disuse atrophy is a particular problem for the elderly, who may already suffer from age-related deficits in muscle function and mass, because such atrophy may lead to permanent disability and premature mortality.

Skeletal muscle atrophy may also result from chronic conditions such as cancer or AIDS cachexia, chronic inflammation, chronic obstructive pulmonary disease (COPD), congestive heart failure, genetic disorders, e.g., muscular dystrophies, neurodegenerative diseases and sarcopenia (age associated muscle loss). In these chronic conditions, skeletal muscle atrophy may lead to premature loss of mobility, thereby adding to the disease-related morbidity.

Little is known regarding the molecular processes that control atrophy or hypertrophy of skeletal muscle. While the triggering event may be different for the various atrophies, several common biochemical changes occur in the affected skeletal muscle fiber, including a decrease in protein synthesis, an increase in protein degradation, and changes in both contractile and metabolic protein isozymes, characteristic of a slow (highly oxidative metabolism/slow contractile protein isoforms) to fast (highly glycolytic metabolism/fast contractile protein isoforms) fiber twitch. Additional changes in skeletal muscle include the loss of vasculature and remodeling of the extracellular matrix. Both fast and slow twitch muscles demonstrate atrophy under the appropriate conditions, with the relative muscle loss depending on the specific atrophy stimuli or condition. Importantly, all these changes are coordinately regulated depending on the changes in physiological and metabolic need.

The processes by which atrophy and hypertrophy occur are conserved across vertebrate species. Multiple studies have demonstrated that the same basic molecular, cellular, and physiological processes occur during atrophy in both rodents and humans. Further, models from different vertebrate species for skeletal muscle atrophy have been successfully utilized to understand and predict human atrophy responses including lower vertebrates like fish and frogs; and mammals like rodents, and humans (See, e.g., Rome, L. R. (2002) Clinical Orthopaedics and Related Research, 403S, S59-S76). For example, atrophy induced by a variety of means in both rodents and humans results in similar changes in muscle anatomy, cross-sectional area, function, fiber type switching, contractile protein expression, and histology. Similarly, Medler compared trends in shortening velocity and force production in skeletal muscles from more than 130 diverse skeletal muscles across vertebrates including insects, crustaceans, mollusks, fish, amphibians, reptiles, birds, and mammals (Medler, S. (2002) Am. J. Physiol. Regulatory Integrative Comp. Physiol. 283, R368-R378). Medler's analysis showed that although differing in size and speed, the skeletal muscle from these diverse species are similar in their physiological properties like shortening velocity and force production. In addition, several agents have been demonstrated to regulate skeletal muscle atrophy in both rodents and in humans. These agents include anabolic steroids, growth hormone, insulin-like growth factor I, and beta-adrenergic agonists. Together, these data demonstrate that skeletal muscle atrophy results from common mechanisms in vertebrates.

Muscular dystrophies encompass a group of inherited, progressive muscle disorders, distinguished clinically by the selective distribution of skeletal muscle weakness. Common forms of muscle dystrophy are Duchenne and Becker dystrophies, each resulting from the inheritance of a mutation in the dystrophin gene. Other dystrophies include limb-girdle muscular dystrophy, fascioscapulohumeral (Landouzy-Dejerine) muscular dystrophy, myotonic dystrophy, and Emery-Dreifuss muscular dystrophy. Current treatment for Duchenne muscular dystrophy includes administration of prednisone (a corticosteroid drug), which while not curative, slows the decline of muscle strength and delays disability. However, corticosteroid treatment may result in skeletal muscle atrophy.

While some agents have been shown to regulate skeletal muscle atrophy and are approved for use in humans for this indication, may of these agents cause one or more undesirable side effects such as hypertrophy of cardiac muscle, neoplasia, hirsutism, androgenization of females, increased morbidity and mortality, liver damage, hypoglycemia, musculoskeletal pain, increased tissue turgor, tachycardia, and edema. Currently, there are no highly effective and selective treatments for either acute or chronic skeletal muscle atrophy. Thus, there is a need to identify target genes that regulate muscle mass or function in order to develop screening methods and to develop novel, effective therapies for muscle atrophy, muscle hypertrophy, and muscular dystrophies.

### SUMMARY OF THE INVENTION

The present invention relates to the use of prostanoid IP receptors to identify compounds that may be useful in the treatment of skeletal muscle atrophy, muscular dystrophy, and/or to induce skeletal muscle hypertrophy by regulating muscle mass or function. The invention also provides in vitro, ex vivo, and in vivo methods for identifying compounds for regulating skeletal muscle mass or function.

In one embodiment, the invention comprises contacting a test compound with a prostanoid IP receptor, determining whether the test compound binds the IP receptor, and selecting those compounds that bind to the prostanoid IP receptor as compounds useful in regulating muscle mass or function.

In another embodiment, the invention comprises contacting a test compound with a cell expressing a prostanoid IP receptor, determining whether the test compound either binds or activates the prostanoid IP receptor, and selecting those compounds that either bind or activate the prostanoid IP receptor as compounds useful for regulating skeletal muscle mass or function.

In another embodiment, the invention provides a method for identifying compounds that regulate muscle mass or function, comprising testing a compound in a skeletal muscle atrophy model system and determining whether the compound regulates skeletal muscle mass or muscle function in the model system.

In another embodiment, the invention provides a method for identifying compounds for regulating skeletal muscle mass or function, comprising administering the compound to a non-human animal and determining whether the compound regulates skeletal muscle mass or muscle function in the animal.

The present invention also relates to the use of prostanoid IP receptor agonists to treat skeletal muscle atrophy, or muscular dystrophy. In particular, the invention provides methods of treating a subject in need thereof, comprising identifying a subject in need of such a treatment, and administering to the subject a safe and effective amount of the prostanoid IP receptor agonist.

The present invention also relates to the use of prostanoid IP receptor antagonists to treat skeletal muscle hypertrophy. In particular, the invention provides methods of treating a subject in need thereof, comprising identifying a subject in need of such a treatment, and administering to the subject a safe and effective amount of the prostanoid IP receptor antagonist. The invention further provides for pharmaceutical compositions comprising a safe and effective amount of a prostanoid IP receptor agonist or antagonist and a pharmaceutically acceptable carrier.

### DESCRIPTION OF SEQUENCE LISTING

Known nucleotide and protein sequences of the prostanoid IP receptors are included in the sequence listing, along with their Genbank accession number(s) and corresponding SEQ ID NOs: in Table 1.

TABLE 1

| | | Genbank | Genbank | | |
|---|---|---|---|---|---|
| Sequence | | Accession No. | Accession No. | SEQ ID NO: | SEQ ID NO: |
| Name | Species | (DNA) | (Protein) | (DNA) | (protein) |
| Prostaglandin | Homo | NM_000960.3 | NP_00095.1 | 1 | 2 |
| I₂ Receptor | sapiens | | | | |
| Prostacyclin | Bos taurus | Z93039 | gi\|1903051 | 3 | 4 |
| Receptor | | | | | |
| Prostacyclin | Rattus | D28966 | gi\|1172502 | 5 | 6 |
| Receptor | norvagicus | | | | |
| Prostacyclin | Rattus | N/A | gi\|1093433 | N/A | 7 |
| Receptor | norvagicus | | | | |
| Prostacyclin | Rattus | XM218457 | gi\|34855233 | 8 | 9 |
| Receptor | norvagicus | | | | |
| Prostacyclin | Mus | D26157 | gi\|627935 | 10 | 11 |
| Receptor | musculus | | | | |
| Prostacyclin | Mus | AY285778 | gi\|3090119 | 12 | 13 |
| Receptor | musculus | | | | |

### DETAILED DESCRIPTION OF THE INVENTION

The present invention relates to the use of prostanoid IP receptors to identify compounds that may be useful in the treatment of skeletal muscle atrophy, muscular dystrophy, and/or to induce skeletal muscle hypertrophy by regulating muscle mass or function. The invention also provides in vitro, ex vivo, and in vivo methods for identifying compounds for regulating skeletal muscle mass or function.

Molecules of the Invention

The invention comprises of various molecules:

genes that are DNA;

transcripts that are RNA;

nucleic acids that regulate their expression such as antisense molecules, siRNAs, micro RNAs;

molecules that may be used to detect them, such as DNA or RNA probes;

primers that may be used to identify and isolate related genes; and

proteins and polypeptides, and compounds that inhibit or activate them.

Thus, the term molecule is used herein to describe all or some of the entities of the invention. It is to be construed in the context it is used.

Prostanoid IP receptors described herein include, but are not limited to, those receptors proteins that bind prostanoid IP with higher affinity than with other prostanoids. Higher or greater affinity indicates that the receptor binds its preferred ligand(s) at lower concentrations than to the other ligands. Selectivity indicates that a receptor binds a preferred ligand over other ligands, but does not imply that the receptor protein would not bind other ligands. This selectivity may be as little as two-fold. Also contemplated herein are natural and non-natural nucleic acid sequences that code for these prostanoid IP receptors. Prostanoid IP receptors have been cloned from various vertebrate species and a non-exhaustive list is included in Table 1 and the Sequence Listing.

Fragments and variants of genes and proteins of the invention are also encompassed by the present invention. By “fragment” is intended a portion of the nucleotide sequence or protein sequence. Fragments may retain the biological activity of the native protein. Fragments of a nucleotide sequence are also useful as hybridization probes and primers or to regulate expression of a gene, e.g., antisense, siRNA, or micro RNA. A biologically active portion may be prepared by isolating a portion of one of the nucleotide sequences of the invention, expressing the encoded portion (e.g., by recombinant expression in vitro), and assessing the activity of the encoded protein.

One of skill in the art would also recognize that genes and proteins from species other than those listed in the sequence listing, particularly vertebrate species, may be useful in the present invention. Such species include, but are not limited to, mice, rats, guinea pigs, rabbits, dogs, pigs, goats, cows, monkeys, chimpanzees, sheep, hamsters, and zebrafish. One of skill in the art would further recognize that by using probes from the known species' sequences, cDNA or genomic sequences homologous to the known sequence could be obtained from the same or alternate species by known cloning methods. Such homologs and orthologs are contemplated to be useful as genes and proteins of the invention.

By “variants” are intended similar sequences. For example, conservative variants may include those sequences that, because of the degeneracy of the genetic code, encode the amino acid sequence of one of the polypeptides of the invention. Naturally occurring allelic variants, and splice variants may be identified with the use of known techniques, e.g., with polymerase chain reaction (PCR), single nucleotide polymorphism (SNP) analysis, and hybridization techniques. In order to isolate orthologs and homologs, generally stringent hybridization conditions are utilized, dictated by specific sequences, sequence length, guanine+cytosine (GC) content, and other parameters. Variant nucleotide sequences also include synthetically derived nucleotide sequences, e.g., derived by using site-directed mutagenesis. Variants may contain additional sequences from the genomic locus alone or in combination with other sequences.

The molecules of the invention also include truncated and/or mutated proteins wherein regions of the protein not required for ligand binding or signaling have been deleted or modified. Similarly, they may be mutated to modify their ligand binding or signaling activities. Such mutations may involve non-conservative mutations, deletions, or additions of amino acids or protein domains. Variant proteins may or may not retain biological activity. Such variants may result from, e.g., genetic polymorphism or from human manipulation. Prostanoid IP Receptors also include lower affinity receptors for PGI₂ and PGI₂ analogs. For example, many of the prostanoid IP receptor ligands also bind the prostanoid FP receptor and the prostanoid EP family of receptors with varying affinities. Similarly, ligands for the EP family of receptors also bind to the prostanoid IP receptors.

Fusions of a protein or a protein fragment to a different polypeptide are also contemplated. Using known methods, one of skill in the art would be able to make fusion proteins of the proteins of the invention; that, while different from native form, would be useful. For example, the fusion partner may be a signal (or leader) polypeptide sequence that co-translationally or post-translationally directs transfer of the protein from its site of synthesis to another site (e.g., the yeast α-factor leader). Alternatively, it may be added to facilitate purification or identification of the protein of the invention (e.g., poly-His, Flag peptide, or fluorescent proteins).

The molecules of the invention may be prepared by various methods, including, but not limited to, cloning, PCR-based cloning, site-directed mutagenesis, mutagenesis, DNA shuffling, and nucleotide sequence alterations known in the art. See, for example, Molecular Cloning: A Laboratory Manual, 2^{nd} Edition, Sambrook, Fristch, and Maniatis (1989), Cold Spring Harbor Laboratory Press; Current Protocols in Molecular Biology, Ausubel et al., (1996) and updates, John Wiley and Sons; Methods in Molecular Biology (series), volumes 158 and 182. Humana Press; PCR Protocols: A guide to Methods and Applications, Innis, Gelfand, Sninsky, and White, 1990, Academic Press.

Libraries of recombinant polynucleotides may also be generated from a population of related sequences comprising regions that have substantial sequence identity and may be recombined in vitro or in vivo. For example, using this approach, sequence motifs encoding a domain of interest may be shuffled between a gene of the invention and other known genes to obtain a new gene coding for a protein with an altered property of interest e.g. a dominant negative mutation (Ohba et al. (1998) Mol. Cell. Biol. 18:51199-51207, Matsumoto et al. (2001) J. Biol. Chem. 276:14400-14406). Strategies for such DNA shuffling are known in the art.

The “percent identity” or “sequence identity” may be determined by aligning two sequences or subsequences over a comparison window, wherein the portion of the sequence in the comparison window may optionally comprise additions or deletions (i.e., gaps) as compared to the reference sequence (which may comprise additions or deletions) for optimal alignment of the two sequences. The percentage is calculated by determining the number of positions at which an identical residue (e.g., nucleic acid base or amino acid) occurs in both sequences, dividing the number of matched positions by the total number of positions in the window of comparison, and multiplying the result by 100 to yield the percentage of sequence identity.

Percentage sequence identity may be calculated by the local homology algorithm of Smith & Waterman, Adv. Appl. Math. 2:482-485 (1981); or by the homology alignment algorithm of Needleman & Wunsch, J. Mol. Biol. 48:443-445 (1970); either manually or by computerized implementations of these algorithms (GAP & BESTFIT in the GCG Wisconsin Software Package, Genetics Computer Group; various BLASTs from the National Center for Biotechnology Information (NCBI), NIH).

A preferred method for determining homology or sequence identity is by BLAST (Basic Local Alignment Search Tool) analysis using the algorithm employed by the programs blastp, blastn, blastx, tblastn, and tblastx (Karlin et al. (1990) Proc. Natl. Acad. Sci. USA 87, 2264-2268 and Altschul, (1993) J. Mol. Evol. 36, 290-300), which are tailored for sequence similarity searching.

As described herein, these various genes and proteins, their allelic and other variants (e.g. splice variants), their homologs and orthologs from other species and various fragments and mutants exhibit sequence variations. These sequences may exhibit at least about 65% sequence identity at the nucleotide level to the genes; preferably at least about 80%, more preferably at least about 85%, more preferably at least about 90%, more preferably at least about 95%, more preferably at least about 98% sequence identity to the genes. With regard to amino acid identity, a high degree of identity is seen to be at least about 50% identity, more preferably at least about 75%, more preferably at least about 85%, more preferably at least about 95%, more preferably at least about 98% sequence identity.

Cell Lines, Vectors, Cloning, and Expression of Recombinant Molecules

Molecules of the invention and their homologs or variants may be prepared for various uses, including, but not limited to: to purify a large quantity of protein or nucleic acid product, to generate antibodies, for use as reagents in the screening assays, and for use in pharmaceutical compositions. Some embodiments may be carried out using an isolated gene or a protein, while other embodiments may be carried out with the use of cells that express them.

Where the source of the molecule is a cell line, the cells may endogenously express it; may have been stimulated to increase endogenous expression; or may have been genetically engineered to express the molecule. Expression of a protein of interest may be determined by, for example, detection of the polypeptide with an appropriate antibody (e.g. Western blot), use of a DNA probe to detect mRNA encoding the protein (e.g., northern blot or various PCR-based techniques), or measuring the binding of an agent selective for the polypeptide of interest (e.g., a suitably-labeled selective ligand).

The present invention further provides recombinant molecules that contain a coding sequence of, or a variant form of, a molecule of invention. In a recombinant molecule, a coding DNA sequence is operably linked to other DNA sequences of interest including, but not limited to, various control sequences for integration, replication, transcription, expression, and modification.

The choice of vector and control sequences to which a gene sequence of the present invention is operably linked depends upon the functional properties desired (e.g., protein expression, the host cell to be transformed). A vector of the present invention may be capable of directing the replication or insertion into the host chromosome, and preferably expression of a gene.

Control elements that are used for regulating the expression of a gene are known in the art and include, but are not limited to, inducible promoters, constitutive promoters, secretion signals, enhancers, termination signals, ribosome-binding sites, and other regulatory elements. Preferably, the inducible promoter is readily controlled, such as being responsive to a nutrient, or an antibiotic.

In one embodiment, the vector containing a coding nucleic acid molecule may include a prokaryotic replicon, i.e., a DNA sequence having the ability to direct autonomous replication and maintenance of the recombinant DNA molecule extra-chromosomally in a prokaryotic host cell, such as a bacterial host cell. In addition, vectors that include a prokaryotic replicon may also include a gene whose expression confers a detectable marker (e.g., resistance to ampicillin).

Vectors may further include a prokaryotic or bacteriophage promoter capable of directing the expression (transcription and translation) of the coding gene sequences in a bacterial host cell, such as E. coli. Promoter sequences compatible with bacterial hosts may be provided in plasmid vectors containing convenient restriction sites for insertion of a DNA segment of the present invention, e.g., pcDNA1, pcDNA3.

Expression vectors compatible with eukaryotic cells may also be used to form a recombinant molecule that contains a sequence of interest. Commercially available vectors often contain both prokaryotic and eukaryotic replicons and control sequences, for easy switch from prokaryotic to eukaryotic cell and ES cells for generating transgenic cells or animals (e.g., pcDNA series from Invitrogen™).

Eukaryotic cell expression vectors used to construct the recombinant molecules of the present invention may further include a selectable marker that is effective in a eukaryotic cell (e.g., neomycin resistance). Alternatively, the selectable marker may be present on a separate plasmid, the two vectors introduced by co-transfection of the host cell, and transfectants selected by culturing in the appropriate drug for the selectable marker. Vectors may also contain fusion protein, or tag sequences that facilitate purification or detection of the expressed protein.

The present invention further provides host cells transformed with a recombinant molecules of the invention. The host cell may be a prokaryote, e.g., a bacterium, or a eukaryote, e.g., yeast, insect or vertebrate cells, including, but not limited to, cells from a mouse, monkey, frog, or human. Commonly used eukaryotic host cell lines include, but are not limited to, CHO cells, ATCC CCL61, NIH-3T3, and BHK cells. Primary cell cultures from animals may also be used.

Transformation of appropriate host cells with a molecule of the present invention may be accomplished by known methods that depend on the host system employed. For transforming prokaryotic host cells, electroporation and salt treatment methods may be employed, while for transformation of eukaryotic cells, electroporation, cationic lipids, or salt treatment methods may be employed (See Sambrook et al. (1989) supra). Viral vectors, including, but not limited to, retroviral and adenoviral vectors have also been developed that facilitate transfection of primary or terminally differentiated cells.

Successfully transformed cells may be cloned to produce stable clones. Cells from these clones may be harvested, lysed and their content examined for the presence of the recombinant molecules using known methods.

Selection of Test Compounds

Compounds that may be screened in accordance with the assays of the invention (described below) include, but are not limited to, libraries of known compounds, including natural products, such as plant or animal extracts. Also included are synthetic chemicals, biological materials, e.g., proteins, nucleic acids, and peptides, including, but not limited to, members of random peptide libraries and combinatorial chemistry derived molecular libraries made of D- or L-configuration amino acids, phosphopeptides, antibodies (including, but not limited to, polyclonal, monoclonal, chimeric, human, anti-idiotypic or single chain antibodies, and Fab, F(ab′)₂ and Fab expression library fragments, and epitope-binding fragments thereof); and other organic and inorganic molecules.

In addition to the traditional sources of test compounds, computer modeling and searching technologies permit the rational selection of test compounds by utilizing structural information from the ligand binding sites of proteins of the present invention. Such rational selection of test compounds may decrease the number of test compounds that must be screened in order to identify a therapeutic compound. Knowledge of the protein sequences of the present invention may allow for generation of models of their binding sites that may be used to screen for potential ligands. This process may be accomplished in manners known in the art. A preferred approach involves generating a sequence alignment of the protein sequence to a template (derived from the crystal structures or NMR-based model of a similar protein(s), conversion of the amino acid structures and refining the model by molecular mechanics and visual examination. If a strong sequence alignment cannot be obtained then a model may also be generated by building models of the hydrophobic helices. Mutational data that point towards contacts residues may also be used to position the helices relative to each other so that these contacts are achieved. During this process, docking of the known ligands into the binding site cavity within the helices may also be used to help position the helices by developing interactions that would stabilize the binding of the ligand. The model may be completed by refinement using molecular mechanics and loop building using standard homology modeling techniques. General information regarding modeling may be found in Schoneberg, T. et. al., Molecular and Cellular Endocrinology, 151:181-193 (1999), Flower, D., Biochimica et Biophysica Acta, 1422:207-234 (1999), and Sexton, P. M., Current Opinion in Drug Discovery and Development, 2(5):440-448 (1999).

Once the model is completed, it may be used in conjunction with one of several computer programs to narrow the number of compounds to be screened by the screening methods of the present invention, e.g., the DOCK program (UCSF Molecular Design Institute, San Francisco, Calif.) or FLEXX (Tripos Inc., St. Louis, Mo.). One may screen databases of commercial and/or proprietary compounds for steric fit and rough electrostatic complementarity to the binding site.

Screening Assays to Identify Compounds

The finding that the genes of the present invention play a role in regulating skeletal muscle mass or function and skeletal muscle atrophy, provides for various methods of screening one or more compounds to identify compounds that ultimately may be used for prophylactic or therapeutic treatment of skeletal muscle atrophy.

When selecting compounds useful for prevention or treatment, it may be preferable that the compounds be selective for proteins of the invention. For initial screening, it may be preferred that the in vitro screen be carried out using a protein of the invention with an amino acid sequence that is, e.g., at least about 80% identical, preferably at least about 90% identical, and more preferably identical to a sequence listed in the sequence listing herein. Preferably, the test compounds may be screened against a vertebrate protein, more preferably a human protein. For screening compounds that ultimately may be used to regulate skeletal muscle mass or function, it may be preferable to use the protein from the species in which treatment is contemplated.

The methods of the present invention may be amenable to high throughput applications; however, use of as few as one test compound in the method is encompassed by the term “screening”. This in vitro screen provides a means by which to select a range of compounds, i.e., the compounds, which merit further investigation. For example, compounds that activate a protein of the invention at concentrations of less than 200 nM might be further tested in an animal model of skeletal muscle atrophy, whereas those above that threshold may not be further tested.

The assay systems described below may be formulated into kits comprising a protein of the invention or cells expressing a protein of the invention, which may be packaged in a variety of containers, e.g., vials, tubes microtitre plates, bottles and the like. Other reagents may be included in separate containers and provided with the kit, e.g., positive and negative control samples, and buffers.

In one embodiment, the invention provides a method to identify compounds that bind to a protein of the invention. Methods to determine binding of a compound to a protein are known in the art. The assays include incubating a protein of the invention with a labeled compound, known to bind to the protein, in the presence or absence of a test compound and determining the amount of bound labeled compound. The source of a protein of the invention may either be cells expressing the protein or some form of isolated protein. The labeled compound may be a known ligand or a ligand analog labeled such that it may be measured, preferably quantitatively (e.g., labeled with ¹²⁵I, ³⁵S methionine, or a fluorescent tag, or peptide or a fluorescent protein fusions). Such methods of labeling are known in the art. Test compounds that bind to a protein of the invention may reduce ligand bound to the protein, thereby reducing the signal level compared to control samples. Variations of this technique have been described Keen, M., Radioligand Binding Methods for Membrane Preparations and Intact cells in Receptor Signal Transduction Protocols, R. A. J. Challis, (ed), Humana Press Inc., Totoway N.J. (1997).

In another embodiment, the invention provides methods for screening test compounds to identify compounds that activate a protein of the invention. The assays may be cell-free or cell-based, which are known to differentiate agonist and antagonist binding. Cell-based assays include contacting cells that express a protein of the invention with a test compound or a control substance and measuring activation of the protein by measuring the expression or activity of components of the affected signal transduction pathways. For example, after contact with a test compound, lysates of the cells may be prepared and assayed for induction of second messengers like cAMP; or transcription, translation, or modification of a protein, e.g., phosphorylation, or glycosylation.

In one embodiment, cAMP induction may be measured with the use of recombinant constructs containing the cAMP responsive element linked to any of a variety of reporter genes. Such reporter genes include, but are not limited to, chloramphenicol acetyltransferase (CAT), luciferase, glucuronide synthetase, growth hormone, fluorescent proteins, or alkaline phosphatase. Following exposure of the cells to a test compound, the level of reporter gene expression may be quantified to determine the test compound's ability to increase cAMP levels and thus determine a test compound's ability to activate a protein of the invention.

In another embodiment, specific phospho-tyrosine or phospho-serine antibodies may be utilized to measure the level of phosphorylation of a signaling protein after the exposure to a test compound, whereby a significant deviation in phosphorylation levels compared to control samples would indicate activation of a protein of the invention. In some instances, a protein's (for example receptor) responses subside, or become desensitized, after prolonged exposure to an agonist. Alternatively, the protein of interest may be an enzyme and thus the effect of the binding of the test compounds could be measured in terms of changes in the enzymatic activity.

Screening of Compounds using Models of Skeletal Muscle Atrophy

Compounds selected from one or more test compounds by an in vitro assay may be further tested for their ability to regulate skeletal muscle mass or function in model systems of skeletal muscle atrophy and/or hypertrophy. Such models of skeletal muscle atrophy or hypertrophy include both in vitro cell culture models and in vivo animal models. Such additional levels of screening may be useful to narrow the range of compounds that merit further investigation.

Cell Culture Models of Muscle Atrophy

In vitro models of skeletal muscle atrophy are known in the art. Such models are described, for example, in Chromiak, J. A., et al., In Vitro Cell. Dev. Biol. Anim., 34(9): 694-703 (1998), Shansky, J., et al., In Vitro Cell. Dev. Biol. Anim., 33(9): 659-661 (1997), Perrone, C. E. et al., J. Biol. Chem. 270(5): 2099-2106 (1995), Chromiac, J. A. and Vandenburgh, H. H., J. Cell. Physiol. 159(3): 407-414 (1994). Cell culture models are treated with compounds and the response of the model to the treatment is measured by assessing changes in muscle markers such as: muscle protein synthesis or degradation, and/or changes in skeletal muscle mass or contractile function. Those compounds that induce sufficient changes may be screened further in an animal model of skeletal muscle atrophy.

Animal Models of Skeletal Muscle Atrophy

The compounds may be administered to non-human animals and the response of the animals is monitored, for example, by assessing changes in markers of atrophy or hypertrophy such as: skeletal muscle mass, skeletal muscle function, muscle or myofiber cross-sectional area, contractile protein content, non-contractile protein content or a biochemical or a genetic marker that correlates with skeletal muscle mass or function changes. In addition to assessing the ability of a compound to regulate skeletal muscle atrophy, undesirable side effects such as toxicity may also be detected in such a screen. The absence of unacceptable levels of side effects may be used as a further criterion for the selection of compounds.

A variety of animal models for skeletal muscle atrophy are known in the art, such as those described in the following references: Appell, H-J. Sports Medicine 10:42-58 (1990), Hasselgren, P-O. and Fischer, J. E. World J. Surg. 22:203-208 (1998), Agbenyega, E. T. and Wareham, A. C. Comp. Biochem. Physiol. 102A:141-145 (1992), Thomason, D. B. and Booth, F. W. J. Appl. Physiol. 68:1-12 (1990), Bramanti, P., et al. Int. J. Anat. Embryol. 103:45-64 (1998), Cartee, G. D. J. Gerontol. A Biol. Sci. Med. Sci. 50:137-141 (1995), and Bloomfield, S. A. Med. Sci. Sports Exerc. 29:197-206 (1997). Preferred animals for these models are mice and rats, but any suitable vertebrate may be used. These models include, for example, models of disuse-induced atrophy, such as casting or otherwise immobilizing limbs, hind limb suspension, complete animal immobilization, and reduced gravity situations. Models of nerve damage induced atrophy include, for example, nerve crush, removal of sections of nerves which innervate specific muscles, toxin application to nerves and infection of nerves with viral, bacterial or eukaryotic infectious agents. Models of glucocorticoid-induced atrophy include application of atrophy-inducing doses of exogenous glucocorticoid to animals, and stimulation of endogenous corticosteroid production, for example, by application of hormones that activate the hypothalamus-pituitary-adrenal axis. Models of sepsis-induced atrophy include, for example, inoculation with sepsis-inducing organisms such as bacteria, treatment with immune-activating compounds such as bacterial cell wall extract or endotoxin, and puncture of intestinal walls. Models of cachexia-induced atrophy include, for example, inoculation with tumorigenic cells with cachexia forming potential, infection with infectious agents that result in cachexia and treatment with hormones or cytokines such as CNTF, TNF, IL-6, or IL-1. Models of heart failure-induced atrophy include the manipulation of an animal so that heart failure occurs with concomitant skeletal muscle atrophy. Neurodegenerative disease-induced atrophy models include autoimmune animal models such as those resulting from immunization of an animal with neuronal components. Muscular dystrophy-induced models of atrophy include natural or genetically induced models of muscular dystrophy such as the mutation of the dystrophin gene that occurs in the Mdx mouse.

Animal models of skeletal muscle hypertrophy include, but are not limited to, models of increased limb muscle use due to inactivation of the opposing limb, reweighing following a disuse atrophy inducing event, reutilization of a muscle which atrophied because of transient nerve damage, increased use of selective muscles due to inactivation of a synergistic muscle (e.g., compensatory hypertrophy), increased muscle utilization due to increased load placed on the muscle and hypertrophy resulting from removal of the glucocorticoid after glucocorticoid-induced atrophy.

The sciatic nerve denervation atrophy model involves anesthetizing the animal followed by the surgical removal of a short segment of either the right or left sciatic nerve, e.g., in mice, the sciatic nerve is isolated approximately at the midpoint along the femur, and a 3-5 mm segment is removed. This denervates the lower hind limb musculature resulting in atrophy of these muscles. The extent of atrophy in the affected muscles is analyzed, for example, by measuring muscle mass, muscle cross-sectional area, myofiber cross-sectional area, or contractile protein content.

The glucocorticoid-induced atrophy model involves the administration of a glucocorticoid to the test animal, e.g., 1.2 mg/kg/day of dexamethasone in the drinking water. The leg casting disuse atrophy model involves casting one hind leg of an animal from the knee down through the foot.

Transgenic Animals and Gene Therapy

Animals of various species, preferably vertebrates, including, but not limited to, mice, rats, rabbits, guinea pigs, pigs, goats, dogs, frogs, and non-human primates may be used to generate transgenic animals expressing the proteins of the invention. Several techniques are known in the art and may be used to introduce transgenes into animals to produce the founder lines of transgenic animals. Such techniques include, but are not limited to, pronuclear microinjection, retrovirus-mediated gene transfer into germ lines, gene targeting in embryonic stem cells, electroporation of embryos and sperm-mediated gene transfer.

The overall activity of a protein of the invention may be increased by overexpressing the gene for that protein. Overexpression tends to increase the total cellular protein activity, thus, regulating skeletal muscle mass or function. The gene or genes of interest are inserted into a vector suitable for expression in the subject. These vectors include, but are not limited to, adenovirus, adenovirus associated virus, retrovirus and herpes virus vectors in addition to other particles that introduce DNA into cells (e.g., liposome, gold particles) or by direct injection of the DNA expression vector, containing the gene of interest, into human tissue (e.g., muscle).

Pharmaceutical Formulations and Methods for Use

Compounds identified by screening methods described herein may be administered to individuals to treat or to prevent diseases or disorders that are regulated by genes and proteins of the invention. The term “treatment” is used herein to mean that administration of a compound of the present invention mitigates a disease or a disorder in a host. Thus, the term “treatment” includes, preventing a disorder from occurring in a host, particularly when the host is predisposed to acquiring the disease, but has not yet been diagnosed with the disease; inhibiting the disorder; and/or alleviating or reversing the disorder. Insofar as the methods of the present invention are directed to preventing disorders, it is understood that the term “prevent” does not require that the disease state be completely thwarted. (See Webster's Ninth Collegiate Dictionary.) Rather, as used herein, the term preventing encompasses to the ability of the skilled artisan to identify a population that is susceptible to disorders, such that administration of the compounds of the present invention may occur prior to onset of a disease. The term does not imply that the disease state be completely avoided. The compounds identified by the screening methods of the present invention may be administered in conjunction with other compounds.

Safety and therapeutic efficacy of compounds identified may be determined by standard procedures using in vitro or in vivo technologies. Compounds that exhibit sufficient therapeutic indices may be preferred, although compounds with otherwise sufficient therapeutic indices may be useful if the level of side effects is acceptable in the population of intended use. The data obtained from the in vitro and in vivo toxicological and pharmacological techniques may be used to formulate the range of doses.

Effectiveness of a compound may further be assessed either in animal models or in clinical trials of patients with, or at risk for, skeletal muscle atrophy by observing the change in skeletal muscle mass, skeletal muscle function, biochemical markers of muscle breakdown or quality of life measures. Methods of measuring skeletal muscle mass in human subjects are known in the art and include, for example, measuring the girth of a limb; measuring muscle thickness with for instance, computer tomography, MRI or supersonics; or muscle biopsy to examine morphological and biochemical parameters (e.g., cross-section fiber area, fiber diameter or enzyme activities). Furthermore, because skeletal muscle mass is correlated with skeletal muscle function, muscle function may be used as a surrogate marker of mass and muscle mass changes may be assessed using functional measurements, e.g., strength, the force of a group of synergist muscles, or contraction characteristics found in electromyographic recordings. In addition, muscle protein loss because of muscle atrophy may be measured by quantitating levels of amino acids or amino acids derivatives, i.e., 3-methyl histidine, in the urine or blood of a subject. See Appell, Sports Med. 10: 42-58 (1990). Quality of life measures include, but are not limited to, the ease of getting out of a chair, number of steps taken before feeling tired, or ability to climb stairs.

As used herein, “pharmaceutically acceptable carrier” is intended to include solvents, dispersion media, coatings, antibacterial and antifungal agents, isotonic and absorption delaying agents, and the like, compatible with pharmaceutical administration. The use of such media and agents for pharmaceutically active substances is known in the art. Except insofar as any conventional media or agent is incompatible with the active compound, such media may be used in the compositions of the invention. Supplementary active compounds may also be incorporated into the compositions. A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application may include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH may be adjusted with acids or bases, such as hydrochloric acid or sodium hydroxide. The parenteral preparation may be enclosed in ampoules, disposable syringes or multiple dose vials made of glass or plastic.

Pharmaceutical compositions suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The composition should be sterile and should be fluid to the extent that easy syringability exists. It should be stable under the conditions of manufacture and storage and should be preserved against the contaminating action of microorganisms such as bacteria and fungi. The carrier may be a solvent or dispersion medium containing, for example, water, ethanol, polyol (for example, glycerol, propylene glycol, and liquid polyethylene glycol, and the like), and suitable mixtures thereof. The proper fluidity may be maintained, for example, by the use of a coating such as lecithin, by the maintenance of the required particle size in the case of dispersion, and by the use of surfactants. Prevention of the microbial growth may be achieved by various antibacterial and antifungal agents, for example, parabens, chlorobutanol, phenol, ascorbic acid, thimerosal, and the like. It may be preferable to include isotonic agents, for example, sugars, polyalcohols such as mannitol, sorbitol, and sodium chloride in the composition. Prolonged absorption of the injectable compositions may be brought about by including in the composition an agent that delays absorption, for example, aluminum monostearate and gelatin.

Sterile injectable solutions may be prepared by incorporating the active compound in the required amount in an appropriate solvent with one or a combination of ingredients enumerated above, as required, followed by filtered sterilization. Dispersion media may be prepared by incorporating the active compound into a sterile vehicle that contains a basic dispersion medium and the required other ingredients. In the case of sterile powders for the preparation of sterile injectable solutions, preferred methods of preparation include vacuum drying and freeze-drying which yields a powder of the active ingredient plus any additional desired ingredient from a previously sterile-filtered solution thereof.

Oral compositions may include an inert diluent or an edible carrier. They may be enclosed in gelatin capsules or compressed into tablets. For oral administration, the agent may be contained in enteric forms to survive the stomach, or further coated or mixed for a release in a particular region of the GI tract by known methods. For the purpose of oral therapeutic administration, the active compound may be incorporated with excipients and used in the form of tablets, troches, or capsules. Oral compositions may also be prepared using a fluid carrier for use as a mouthwash, wherein the compound in the fluid carrier is applied orally and swished and expectorated or swallowed. Pharmaceutically compatible binding agents, and/or adjuvant materials may be included as part of the composition. The tablets, pills, capsules, troches and the like may contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

For administration by inhalation, the compounds may be delivered in the form of an aerosol spray from pressured container or dispenser, which contains a suitable propellant, e.g., a gas such as carbon dioxide, or a nebulizer.

Systemic administration may also be by transmucosal or transdermal means. For transmucosal or transdermal administration, penetrants appropriate to the barrier to be permeated may be used in the formulation. Such penetrants are generally known in the art, and include, for example, for transmucosal administration, detergents, bile salts, and fusidic acid derivatives. Transmucosal administration may be accomplished using nasal sprays or suppositories. For transdermal administration, the active compounds may be formulated into ointments, salves, gels, or creams as generally known in the art.

The compounds may also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, and polylactic acid. Methods for preparation of such formulations will be apparent to those skilled in the art. Liposomal suspensions may also be used as pharmaceutically acceptable carriers. These may be prepared according to methods known in the art, for example, as described in U.S. Pat. No. 4,522,811.

It may be advantageous to formulate oral or parenteral compositions in dosage unit form for ease of administration and uniformity of dosage. “Dosage unit form” as used herein refers to physically discrete units suited as unitary dosages for the subject to be treated, each unit containing a predetermined quantity of active compound calculated to produce the desired therapeutic effect in association with a pharmaceutical carrier. The specification for the dosage unit forms of the invention may be dictated by and may be dependent on the characteristics of the active compound and the particular therapeutic effect to be achieved, and the limitations inherent in the art of preparing such an active compound for the treatment of animals.

### EXAMPLES

### Example 1

The role of the prostanoid IP receptor in vivo is investigated in the mouse casting model of skeletal muscle atrophy using the pharmacological agents carbaprostacyclin, ciprostene, beraprost and iloprost (Cayman Chemical Company, Ann Arbor, Mich.) which are selective agonists for the prostanoid IP receptor.

Eight mice are used per treatment group in the study described. Following casting of the right hind leg, male mice are injected subcutaneously in the midscapular region once daily, with either carbaprostacyclin, ciprostene, beraprost, iloprost or vehicle control (phosphate buffered saline), for fourteen days at the daily delivered dose indicated. On day fourteen, the tibialis anterior and medial gastrocnemius muscles are removed and weighed to determine the degree of atrophy. Percent inhibition of atrophy is calculated using the following formula:

$$\% Inhibition of Muscle Atrophy = \frac{treated , casted muscle - control , casted muscle}{control , uncasted muscle - control , casted muscle}$$

Statistical significance of the results is determined using ANCOVA (Douglas C. Montgomery, Design and Analysis of Experiments, John Wiley and Sons, New York (2^{nd} ed. 1984).

Table 2. Percent inhibition of casting-induced atrophy of the tibialis anterior and medial gastrocnemius muscles in the mouse following treatment with carbaprostacyclin, ciprostene, beraprost, or iloprost.

TABLE 2

| | | | % inhibition of atrophy | |
|---|---|---|---|---|
| | Chemical | Dose | tibialis | medial |
| | Abstract Service | (mg/ | anterior | gastrocnemius |
| Compound | (CAS) Registry # | kg/d) | muscle | muscle |
| Carbaprostacyclin | 69552-46-1 | 0.3 | 0 | 1 |
| | | 1.0 | 12 | 24* |
| | | 3.0 | 8 | 43* |
| Ciprostene | 81703-55-1 | 0.3 | 5 | 25* |
| | | 1.0 | 9 | 20 |
| | | 3.0 | 25* | 43* |
| Beraprost | 88475-69-8 | 0.3 | 28* | 46* |
| | | 1.0 | 27* | 22 |
| | | 3.0 | 5 | 28* |
| Iloprost | 78919-13-8 | 0.3 | 18 | 42* |
| | | 1.0 | 21* | 36* |
| | | 3.0 | 11 | 31* |

The symbol (*) indicates statistically significant response at p < 0.05.

### Example 2

Construction of vectors that express a prostanoid IP receptor The cDNA sequence for a gene of the present invention is retrieved from suitable public database and two oligonucleotides including one containing the 5′ end of the gene (5′ oligonucleotide) and one containing the 3′ end of the gene (3′ oligonucleotide) are synthesized. Using the above 5′ and 3′ oligonucleotides, the cDNA is amplified by PCR from a suitable animal cDNA library available commercially using a PCR kit. The PCR product is purified and cloned into a vector (e.g. pIRESneo vector (Clonetech Inc., Palo Alto, Calif., USA) using a commercially available PCR cloning kit according to the manufacturer's recommendations. The cloned gene is then used to transform competent E. coli cells. Plasmid DNA is isolated and the insert from at least one clone is sequenced to ensure that the gene sequence is correct. Suitable vertebrate cells (e.g. HEK293 cells containing a stably integrated Mercury CRE-LUC plasmid (Clonetech Inc., Palo Alto, Calif., USA) are transfected with purified plasmid DNA. Cells stably transfected with plasmid DNA are selected by culturing the cells in G418. The stably transfected cells are propagated in DMEM (Life Technologies, Rockville, Md.) containing 10% fetal bovine serum at 37° C. in a 5% CO₂. The clones are then characterized to ensure they have the correct gene activity. Cells expressing the gene at an appropriate level may then be utilized for further analysis.

### Example 3

### Binding Assays

Binding analysis of compounds is performed in whole cells by plating cells from Example 2 expressing a gene of the present invention in a 96 well plate. Cells are seeded in DMEM medium containing 10% fetal bovine serum and incubated at 37° C. in a 5% CO₂ incubator overnight. The culture medium is removed and the appropriate amount of suitably labeled compound is added. The cells are incubated for 90 minutes at room temperature and then washed 4 times with phosphate buffered saline. Following the final wash, the plate is analyzed for binding. For saturation binding analysis, log doses of a compound ranging from 10⁻¹² to 10⁻³ M are added to the cells and binding analyzed both in the absence and in the presence of a saturating concentration of unlabeled compound for evaluation of non-specific binding. The binding analysis may differentiate various compounds based on the binding affinities.

### Example 4

### Activation Assay

Activation analysis is performed by seeding cells from Example 2 into Packard View Plate-96 (Packard Inc., CA). Cells are seeded in DMEM medium containing 10% fetal bovine serum and incubated at 37° C. in a 5% CO₂ incubator overnight. The medium is removed and replaced with DMEM with 0.01% bovine albumin fraction V containing the compound of interest. The cells are further incubated for four hours at 37° C. after which the medium is removed and the cells are washed twice with Hanks Balanced Salt Solution (HBSS). Lysis Reagent is then added to the washed cells and incubated for 20 minutes at 37° C. The cells are then placed at −80° C. for 20 minutes followed by a 20-minute incubation at 37° C. After this incubation, Luciferase Assay Buffer and Luciferase Assay Substrate (Promega Inc., Madison, Wis.) are added to the cell lysates and luciferase activity quantitated using a luminometer. Relative activity of a compound is evaluated by comparing the increase following exposure to compound to the level of luciferase in HEK cells that contain the CRE-LUC construct without the gene of interest following exposure to compound. Specificity of response is also checked by evaluating the luciferase response of hCR/CRE-LUC HEK cells to compound in the presence and absence of a 10-fold excess of an antagonist.

### Example 5

### Determination of Absolute Force Measurement of a Muscle

The extensor digitorum longus (EDL) and soleus muscles are removed tendon-to-tendon from the casted mouse leg. A silk suture is tied to each tendon of the isolated muscles and the muscles are placed into a Plexiglas chamber filled with suitable buffer, constantly bubbled with 95% oxygen/5% carbon dioxide maintained at 25° C. Muscles are aligned horizontally between a servomotor lever arm (Model 305B-LR, Cambridge Technology Inc., Watertown Mass., USA) and the stainless steel hook of a force transducer (Model BG-50; Kulite Semiconductor Products Inc., Leonia N.J., USA) and field stimulated by pulses transmitted between two platinum electrodes placed longitudinally on either side of the muscle. Square wave pulses (0.2 ms duration) generated by a personal computer with a Labview board (Model PCI-MIO 16E-4, Labview Inc., Austin, Tex., USA) are amplified (Acurus power amplifier model A25, Dobbs Ferry, N.Y., USA) to increase tetanic contraction. Stimulation voltage and muscle length (Lo) are adjusted to obtain maximum isometric twitch force. Maximum tetanic force production (Po) is determined from the plateau of the frequency-force relationship.

Except as otherwise noted, all amounts including quantities, percentages, portions, and proportions, are understood to be modified by the word “about”, and amounts are not intended to indicate significant digits.

Except as otherwise noted, the articles “a”, “an”, and “the” mean “one or more”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

What is claimed is:

1. A method for identifying compounds for regulating skeletal muscle mass or function, comprising:
(a) contacting a compound with a prostanoid IP receptor;
(b) determining whether the compound binds the prostanoid IP receptor;
(c) selecting those compounds that bind the prostanoid IP receptor and further determining whether those compounds modulate muscle mass or function in a skeletal muscle atrophy model system; and
(d) identifying those compounds that modulate muscle mass or function in a skeletal muscle atrophy model system as compounds for regulating skeletal muscle mass or function.

2. The method of claim 1, further comprising: administering a compound identified in step (d) of claim 1 to a non-human animal, and determining whether the test compound regulates skeletal muscle mass or function in the animal, wherein test compounds that regulate skeletal muscle mass or function in the animal are identified as compounds for regulating skeletal muscle mass or function.

3. The method of claim 1, wherein the amino acid sequence of the prostanoid IP receptor involved in regulating skeletal muscle mass or function is at least about 80% identical to the amino acid sequence of SEQ ID NO: 2.

4. The method of claim 3, wherein the amino acid sequence of the prostanoid IP receptor involved in regulating skeletal muscle mass or function is at least about 90% identical to the amino acid sequence of SEQ ID NO: 2.

5. The method of claim 1, wherein the amino acid sequence of the prostanoid IP receptor involved in regulating skeletal muscle mass or function is selected from the group consisting of SEQ ID NOs: 2, 4, 6, 7, 9, 11, and 13.

6. A method for identifying compounds for regulating skeletal muscle mass or function, comprising:
(a) contacting a compound with a cell population expressing a prostanoid IP receptor protein;
(b) contacting the test compound with a cell population that does not express the prostanoid IP receptor protein;
(c) determining and comparing the activity of the prostanoid IP receptor protein in the two cell populations;
(d) selecting those compounds that modulate the activity of the prostanoid IP receptor in the cell population that express prostanoid IP receptor protein compared to the activity in the cell population that does not express prostanoid IP receptor protein for further testing;
(e) further determining whether compounds selected in step (d) modulate muscle mass or function in a skeletal muscle atrophy model system; and
(f) identifying those compounds that modulate muscle mass or function in a skeletal muscle atrophy model system as compounds for regulating skeletal muscle mass or function.

7. The method of claim 6, further comprising: administering a compound identified in step (f) of claim 6 to a non-human animal, and determining whether the test compound regulates skeletal muscle mass or function in the animal, wherein test compounds that regulate skeletal muscle mass or function in the animal are identified as compounds for regulating skeletal muscle mass or function.

8. The method of claim 6, wherein the amino acid sequence of the prostanoid IP receptor involved in regulating skeletal muscle mass or function is at least about 80% identical to the amino acid sequence of SEQ ID NO: 2.

9. The method of claim 8, wherein the amino acid sequence of the prostanoid IP receptor involved in regulating skeletal muscle mass or function is at least about 90% identical to the amino acid sequence of SEQ ID NO: 2.

10. The method of claim 6, wherein the amino acid sequence of the prostanoid IP receptor involved in regulating skeletal muscle mass or function is selected from the group consisting of SEQ ID NOs: 2, 4, 6, 7, 9, 11, and 13.

11. A method for identifying compounds for regulating skeletal muscle mass or function, comprising: determining whether a compound modulates muscle mass or function in a skeletal muscle atrophy model system; and identifying those compounds that modulate muscle mass or function in a skeletal muscle atrophy model system as compounds for regulating skeletal muscle mass or function.

12. A pharmaceutical composition, comprising:
(a) a safe and effective amount of a prostanoid IP receptor agonist; and
(b) a pharmaceutically acceptable carrier.

13. A pharmaceutical composition according to claim 11, wherein the prostanoid IP receptor agonist is carbaprostacyclin.

14. A pharmaceutical composition according to claim 11, wherein the prostanoid IP receptor agonist is ciprostene.

15. A pharmaceutical composition according to claim 11, wherein the prostanoid IP receptor agonist is beraprost.

16. A pharmaceutical composition according to claim 1, wherein the prostanoid IP receptor agonist is iloprost.

17. A pharmaceutical composition comprising a safe and effective amount of a compound identified by the method according to claim 1.

18. A pharmaceutical composition comprising a safe and effective amount of a compound identified by the method according to claim 6.

19. A method for modulating skeletal muscle mass or function in a subject in which such a modulation is desirable, comprising:
(a) identifying a subject in which an increase in muscle mass or function is desirable; and
(b) administering to the subject a safe and effective amount of compound that is a prostanoid IP receptor agonist.

20. The method according to claim 18 for modulating skeletal muscle mass or function in a subject in which such a modulation is desirable, wherein the compound is selected from the group consisting of carbaprostacyclin, ciprostene, beraprost and iloprost.


SEQUENCE LISTING (13 sequences)

SEQ ID NO: 1 - DNA, 2103 bp, homo sapiens
  CDS (114)..(1274)
aagctgacacacagaccgacacaggcagcgagagacacgaggagcaaagcaagtgaaggcacagacgcacgggacaggagagcctgggcaagactggagagcccagacctgggatggcggattcgtgcaggaacctcacctacgtgcggggctcggtggggccggccaccagcaccctgatgttcgtggccggtgtggtgggcaacgggctggccctgggcatcctgagcgcacggcgaccggcgcgcccctcggccttcgcggtgctggtgaccggactggcggccaccgacctgctgggcaccagcttcctgagcccggccgtgttcgtggcctatgcgcgcaacagctccctgctgggcctggcccgaggcggccccgccctgtgcgatgccttcgccttcgccatgaccttcttcggcctggcgtccatgctcatcctctttgccatggccgtggagcgctgcctggcgctgagccacccctacctctacgcgcagctggacgggccccgctgcgcccgcctggcgctgccagccatctacgccttctgcgtcctcttctgcgcgctgcccctgctgggcctgggccaacaccagcagtactgccccggcagctggtgcttcctccgcatgcgctgggcccagccgggcggcgccgccttctcgctggcctacgccggcctggtggccctgctggtggctgccatcttcctctgcaacggctcggtcaccctcagcctctgccgcatgtaccgccagcagaagcgccaccagggctctctgggtccacggccgcgcaccggagaggacgaggtggaccacctgatcctgctggccctcatgacagtggtcatggccgtgtgctccctgcctctcacgatccgctgcttcacccaggctgtcgcccctgacagcagcagtgagatgggggacctccttgccttccgcttctacgccttcaaccccatcctggacccctgggtcttcatccttttccgcaaggctgtcttccagcgactcaagctctgggtctgctgcctgtgcctcgggcctgcccacggagactcgcagacacccctttcccagctcgcctcagggaggagggacccaagggccccctctgctcctgtgggaaaggaggggagctgcgtgcctttgtcggcttggggcgaggggcaggtggagcccttgcctcccacacagcagtccagcggcagcgccgtgggaacgtcgtccaaagcagaagccagcgtcgcctgctccctctgctgacatttcaagctgaccctgtgatctctgccctgtcttcgggcgacaggagccagaaaatcagggacatggctgatggctgcggatgctggaaccttggcccccaaactctggggccgatcagctgctgtttctcctgcggcagggcagtcgctgctggctctgggaagagagtgagggacagaggaaacgtttatcctggagtgcagaaagaatggttctctcaaaataaccagtggcctggccgacctgctctggccctggattccccatccatctcattgtctaaatatttagaaggcggagaagttcccagaggcttctgtacagtcaggtctgctctggtctgggtgctggctccaatctgcgtccacttaggaggcccaactgcccaccccaagtccccaggggatggccctccccctctaccaagccactccaagagccagcccctttctgctccacaaaaaccacagttattggaaaagctccctgccttcccttgccgctggtcccccaccaggcttgggagccctggcatcccaaaggggcaacgggaggaaggggaggctgctgcattgtgggtgatgacgtaggacatgtgcttggtacaaaaagggcctgagacattccacctagcttgactggctgcaagatgagaactgggggggtgcaggtggtggggagacagatggagaagctggcagatgaagggtgggggctgcggatcccagggactgccccagaacacaaacctaagtcctgtgcctgtccccagggtcctgaataaataaaagcctccttgcagagcctgaaaaaaaaaaaaaaaaaa

SEQ ID NO: 2 - protein, 386 aa, homo sapiens [sequence omitted]

SEQ ID NO: 3 - DNA, 1248 bp, Bos taurus
  CDS (81)..(1238)
tccctcgacacctcccaccctccacctcccgggaaccccctccagccccccctcccccgggaggcagccggaggcccgggatggcagattcgtgccggaacctcacgtacgtgcgggactcggtgggcccggccaccagcaccctgatgttcgtggcgggcgtggtgggcaacgggctggcactgggcatcttgggggcgcggcggcattcgcgcccctcggccttcgctgtgctggtcaccgggctgggggtcaccgacctgctgggcacgtgcttcctgagcccagcagtgttcgccgcctacgcccgcaacagctcgctgctgggccttgcccggggccgccccgcgctgtgcgacgccttcgccttcgccatgaccttcttcggcctggcctccacgctcatcctcttcgccatggccgtggagcgctgcctggcgctcagccacccctacctctacgcccagctggacggaccccgccgcgcccgcctggcgctgccggccatctacgccttctgcacgatcttctgctcgctgcccttcctgggcctgggccaacaccagcagtactgccccggcagctggtgcttcatccgcatgcgctcggccgagccgggcggctgcgccttcctgctggcgtacgccagcctcgtggccctgctggtggccgccatcgtcctctgcaacggctccgtcaccctcagcctttgccgcatgtaccgccagcagaggcgccaccaggctcgctgtccccggccccgggcaggcgaggacgaggttgaccacttgattctgctggctctcatgacgggcatcatggctgtgtgctccctgcctcttacccctcagatccgcggcttcacccaggccatcgccccggacagcagtgagatgggagacctcctggccttccgtttcaatgccttcaaccccattctggatccctgggtctttatcctcttccgcaagtccgtcttccagcggctgaagctctggttctgttgcctgtactcgaggcctgcccaaggcgactctcggacgtccctctctcagtccgcctcagggaggaaggactcaagtgccccccctgctcttgaggggaaaaaggggaactgggtgcctttgtcagcttggggcgaggggcagggcgggcccttaccggctgtacagctacccaccagtaccgtgggaacaccctccaaagcggggtctgaggcagcctgctccctctgctgacatctgcctg

SEQ ID NO: 4 - protein, 385 aa, Bos taurus
MADSCRNLTYVRDSVGPATSTLMFVAGVVGNGLALGILGARRHSRPSAFAVLVTGLGVTDLLGTCFLSPAVFAAYARNSSLLGLARGRPALCDAFAFAMTFFGLASTLILFAMAVERCLALSHPYLYAQLDGPRRARLALPAIYAFCTIFCSLPFLGLGQHQQYCPGSWCFIRMRSAEPGGCAFLLAYASLVALLVAAIVLCNGSVTLSLCRMYRQQRRHQARCPRPRAGEDEVDHLILLALMTGIMAVCSLPLTPQIRGFTQAIAPDSSEMGDLLAFRFNAFNPILDPWVFILFRKSVFQRLKLWFCCLYSRPAQGDSRTSLSQSASGRKDSSAPPALEGKKGNWVPLSAWGEGQGGPLPAVQLPTSTVGTPSKAGSEAACSLC

SEQ ID NO: 5 - DNA, 1360 bp, Rattus norvegicus
  CDS (83)..(1333)
ctggagaagacggaaacaaaagacagaaacacacgcacacggggaactgggagttgactggcagatcccagctgccctgaggatggtggccagcggtggacgtcctgatggtcccccttctatcacccccgagagccctctcattgtaggtggcagagagtggcagggtatggcaggttcttgctggaacatcacctacgttcaagactcggtaggacctgccaccagcaccctgatgtttgtggctggtgtggtgggcaacgggctagcactgggcatcctgggtgcccgacggaggtcacacccatcggcgtttgcagtgttggtcactgggctggcagtgacggatctgctgggcacgtgcttcttgagccccgcggtgtttgtggcctatgctcgaaacagctctttgctgggcctggcccacggtgggacgatgctgtgtgacactttcgccttcgctatgactttcttcggcctggcctccacgctcatcctcttcgccatggctgtggagcggtgcctggctctcagtcatccctacctgtacgcccagctggacgggccccgctgtgcccgcttggctttgcctgccatctacgctttctgttgtctcttctgctcactgcccctgctgggcctgggcgagcaccagcagtattgtcccgggagctggtgcttcatccgcatgcgttccccccagcctggtggctgtgccttctccctggcctatgccagtctcatggccctgctggtgacctccatcttcttctgcaacggctccgtcactctcagcctctgtcacatgtaccgccaacagagacggcaccatggctcgtttgtgccgacctctcgggcacgagaggatgaagtttaccacctgattctgctggccctcatgacaggcatcatggccgtatgctccctgcctctcacgatcagaggattcacgcaggccatcgccccagacagcagagagatgggggacctccacgccttccgttttaatgccttcaaccccatcctggacccctgggtcttcatccttttccgaaaggctgtcttccaacgcctcaagttctggttgtgttgtctgtgtgcccgttctgtccatggggatctgcagacgcccctctcccggcccgtgtcggggagaagagacacactggctcccgattctctccaggctaaggaagggaactgggtgcccctgtcaacctggggtacggggcaggtggcaccattgactgctgtgcctctgtctggtggtgatggctgttctgtgggaatgccatccaaaacagaggctgtggttgcctgctccctctgctgatacctaagctggtcctgcttgccatcc

SEQ ID NO: 6 - protein, 416 aa, Rattus norvegicus
MVASGGRPDGPPSITPESPLIVGGREWQGMAGSCWNITYVQDSVGPATSTLMFVAGVVGNGLALGILGARRRSHPSAFAVLVTGLAVTDLLGTCFLSPAVFVAYARNSSLLGLAHGGTMLCDTFAFAMTFFGLASTLILFAMAVERCLALSHPYLYAQLDGPRCARLALPAIYAFCCLFCSLPLLGLGEHQQYCPGSWCFIRMRSPQPGGCAFSLAYASLMALLVTSIFFCNGSVTLSLCHMYRQQRRHHGSFVPTSRAREDEVYHLILLALMTGIMAVCSLPLTIRGFTQAIAPDSREMGDLHAFRFNAFNPILDPWVFILFRKAVFQRLKFWLCCLCARSVHGDLQTPLSRPVSGRRDTLAPDSLQAKEGNWVPLSTWGTGQVAPLTAVPLSGGDGCSVGMPSKTEAVVACSLC

SEQ ID NO: 7 - protein, 413 aa, Rattus norvegicus
MVASGGRPDGPPSITPESPLIVGGREWQGMAGSCWNITYVQDSVGPATSTLMFVAGVVGNGLALGILGARRRSHPSAFAVLVTGLAVTDLLGTCFLSPAVFVAYARNSSLLGLAHGGTMLCDTFAFAMTFFGLASTLILFAMAVERCLALSHPYLYAQLDGPRCARLALPAIYAFCCLFCSLPLLGLGEHQQYCPGSWCFIRMRSPQPGGCAFSLAYASLMALLVTSIFFCNGSVTLSLCHMYRQQRRHHGSFVPTSRAREDEVYHLILLALMTGIMAVCSLPLTIRGFTQAIAPDSREMGDLHAFRFNAFNPILDPWVFILFRKAVFQRKFWLCCLCARSVHGDLQTPLSRPVSGRRDLAPDSLQAKEGNWVPLSTWGTGQVAPLTAPLSGGDGCSVGMPSKTEAVVACSLC

SEQ ID NO: 8 - DNA, 896 bp, Rattus norvegicus
  CDS (255)..(896)
ggactgagggacttcagaaggcagaggcgccggaggagggtccagaaaccagaaatgtaaaggaggtggcagagaggcttagaggaagggaccggggcaagggacagggaacactgaggcacaaagggacaaggactgacagagaaggaaagaaacaggagagggacagacactggagaagacggaaacaaaagacagaaacacacgcacacggggaactgggagttgactggcagatcccagctgccctgaggatggtggccagcggtggacgtcctgatggtcccccttctatcacccccgagagccctctcattgtaggtggcagagagtggcagggtatggcaggttcttgctggaacatcacctacgttcaagactcgagacggcaccatggctcgtttgtgccgacctctcgggcacgagaggatgaagtttaccacctgattctgctggccctcatgacaggcatcatggccgtatgctccctgcctctcacgatcagaggattcacgcaggccatcgccccagacagcagagagatgggggacctccacgccttccgttttaatgccttcaaccccatcctggacccctgggtcttcatccttttccgaaaggctgtcttccaacgcctcaagttctggttgtgttgtctgtgtgcccgttctgtccatggggatctgcagacgcccctctcccggcccgtgtcggggagaagagacacactggctcccgattctctccaggctaaggaagggaactgggtgcccctgtcaacctggggtacggggcaggtggcaccattgactgctgtgcctctgtctggtggtgatggctgttctgtgggaatgccatccaaaacagaggctgtggttgcctgctccctctgctga

SEQ ID NO: 9 - protein, 213 aa, Rattus norvegicus
MVASGGRPDGPPSITPESPLIVGGREWQGMAGSCWNITYVQDSRRHHGSFVPTSRAREDEVYHLILLALMTGIMAVCSLPLTIRGFTQAIAPDSREMGDLHAFRFNAFNPILDPWVFILFRKAVFQRLKFWLCCLCARSVHGDLQTPLSRPVSGRRDTLAPDSLQAKEGNWVPLSTWGTGQVAPLTAVPLSGGDGCSVGMPSKTEAVVACSLC

SEQ ID NO: 10 - DNA, 3289 bp, Mus musculus
  CDS (281)..(1354)
gaattcgcggccgctcacagactcgcacacagacaggagagactgagggacttcagaaggcagaggtgctggagggtctagaaagcagaaatgtcaaggaggtgacagagaagtgtagaggaaagaacagggggtaggggacagggaacactgaggcacaaagggacaaggacctgacagagaaggaaagaaacaggagagggacagacacaggagaagacagaaacaaaagacagaaacacacgggcaactgggaattgactggcagatcccagatcctatgaagatgatggccagcgatggacatcctggacccccttctgtcactcctgggagccctctcagtgcaggtggcagagagtggcagggcatggcaggctcctgctggaatatcacctacgttcaagactcggtaggacctgccaccagcacgctgatgttcgtggctggtgtagtgggcaacgggctggcattgggcatcctgggtgcccgacggaggtcacacccatcggcctttgcggtgttggtcactgggctggcggtgacggatctgctgggcacgtgcttcttgagccctgcagtgtttgtggcctatgctcgaaacagctctctgctgggcctggcccacggtgggacgatgctgtgtgacactttcgccttcgccatgacgttcttcggcctggcctccacgctcatcctctttgccatggctgtggagcggtgcctggcactcagtcacccctacctgtacgcccagctggacgggccgcgctgcgcccgctttgccttgccttccatctacgctttctgctgcctcttctgctcactgcccctgctgggcctgggcgagcatcagcagtattgcccagggagctggtgcttcatccgcatgcgttccgcccagcctggcggctgtgccttctccctggcctacgccagtctcatggccctgttggtgacctccatcttcttctgcaacggctccgtcactctcagcctctatcacatgtaccgccaacagagacgccaccatggctcgtttgtaccgacctctcgggcacgagaggatgaagtttaccacctgattctgctggccctcatgaccgtcatcatggccgtgtgctccctgcctctcatgatccgaggcttcactcaggccatcgccccagacagcagagagatgggggacctcctcgccttccgcttcaacgccttcaaccccatcctggacccctgggtcttcatccttttccgaaaggctgtcttccaacgcctcaagttctggttgtgttgcctgtgtgcccgttctgtccatggggatttgcaggcgcccctttccagacctgcatcggggagaagagacccaccggctcctacttctctccaggctaaggaagggagctgggtgcccctgtcgtcctggggcacgggacaggtggcaccattgactgctgtgcctctgactggtggtgacggctgctctgtgggaatgccatccaagtcagaggccatcgctgcctgctccctctgctgatatctgagctggtcctgcatgtcatcctaagcccgctgtcacaggaccagaagccagaagatcagggactcaactgacggtagcagaaaccacatatttgatccccagagtctgggggtgtctgctgctgcttctcctccttcggtatggtggctagaggccttggaaagagggtgagggatgccgaaggttctatggctctcaagaaggattctctcaaaataaccagggcctgggcaggctgctctggcccctgggctcctccatccatctctctgtctaaatatttagggagcagaaaaattcccagaggcttctgtggactcgagtctactggggttagagtgtctgctctcctcttccactgagggggcccctggctgcccaccccaggtccccaggggacacaaggcattccagcaacttctgcagaaatgtgtctgcctcccccatttcccatcccctatctgacttggaaatcttgaacccctaagaggaagtgagaagaaaggagggccactgcactgcaggtgacattgctggacacatgcctggcacagaaatggtctgagaaatgccacagctatcttggggaactgcgctgtgtgaactttggaggagggcacagggacaaggcggagatggtggtgatgtagcatgcagaggctccaaaggacttcacaggcatgcttcctaggatgcatgtcccaagcctgtgcccccatcctggaacaacttactgccacctttcagaactggaccacaactcaccatctttggaatctccacaaaagtttccctcattcattcaacaaactcccgggggaccctgaagtcagcaggcactcactcttcctccctcggatcagggtcgcagaaaagtcccaggcctccaagcaaagaatcctgggtgcgtggaaatgagctgcctggcatgaggggaagtgagtcagctggagggccgggctgcatgcgggagggcaagccagtgctcctgctgatgtttctcctggttcctgtttctcagccactccggtcattcgtcctcccactccatctgctcggcagcatattagcaaagcctctccagagaaagtggaaccagggcccagagagggcaaagcccaagcttggaccacacagcagaactttctcacggcctctctgtgcctcagtttcctcagctctaatctgacaggggtgtcttgaggagtaaaggactctctcccaaaagaacactttaacacaggacctggaaggaagccacagagggtgttactgttactgctgtcaatattgttatctgggcaaacatagaaacaaaaaactagaaaataaggcaaggatttctcctgagtttttttttttctcccaagtaagtatatttccatcacactcagaaaaagcaaagagaatcgggagaagagaaaacaggcagaggtaagctgggtgtggaggtgtgtgccacaacgcctcggcccttcttcaggcaggaggtcagagttcaaggtcatcctccactacatacagggttcaaggtcagcctggggtatttgagactctcaaaaccaaatgacaacaacagataaaaccacaaaacaaaccagtctgagttgaccagtcagtcctcaggtactgaaagatactatctgatgccagttgctgactttgtaaatgatccaaataccactgaatggataatttgctgtgaatgataaatggttaaaatggtaatttttaagcggccgcgaattc

SEQ ID NO: 11 - protein, 358 aa, Mus musculus
MKMMASDGHPGPPSVTPGSPLSAGGREWQGMAGSCWNITYVQDSVGPATSTLMFVAGVVGNGLALGILGARRRSHPSAFAVLVTGLAVTDLLGTCFLSPAVFVAYARNSSLLGLAHGGTMLCDTFAFAMTFFGLASTLILFAMAVERCLALSHPYLYAQLDGPRCARFALPSIYAFCCLFCSLPLLGLGEHQQYCPGSWCFIRMRSAQPGGCAFSLAYASLMALLVTSIFFCNGSVTLSLYHMYRQQRRHHGSFVPTSRAREDEVYHLILLALMTVIMAVCSLPLMIRGFTQAIAPDSREMGDLLAFRFNAFNPILDPWVFILFRKAVFQRLKFWLCCLCARSVHGDLQAPLSRPASG

SEQ ID NO: 12 - DNA, 464 bp, Mus musculus
  CDS (3)..(464)
tgtttgtggcctatgctcgaaacagctctctgttgggcttggcccacggtgggacgatgctgtgtgacactttcgccttcgccatgacgttcttcggcctggcctccacgctcatcctctttgccatggctgtggagcggtgcctggcactcagtcacccctacctgtacgcccagctggacgggccgcgctgcgcccgctttgccttgccttccatctacgctttctgctgcctcttctgctcactgcccctgctgggcctgggcgagcatcagcagtattgcccagggagctggtgcttcatccgcatgcgttccgcccagcctggcggctgtgccttctccctggcctacgccagtctcatggccctgttggtgacctccatcttcttctgcaacggctccgtcactctcagcctctatcacatgtaccgccaacagagacgccaccatggctcgtttgta

SEQ ID NO: 13 - protein, 154 aa, Mus musculus
FVAYARNSSLLGLAHGGTMLCDTFAFAMTFFGLASTLILFAMAVERCLALSHPYLYAQLDGPRCARFALPSIYAFCCLFCSLPLLGLGEHQQYCPGSWCFIRMRSAQPGGCAFSLAYASLMALLVTSIFFCNGSVTLSLYHMYRQQRRHHGSFV